(19) United States
(12) Patent Application Publication
(10) Pub. No.: US 2023/0172958 A1
(43) Pub. Date: Jun. 8, 2023

(54) USE OF COMPOUNDS FOR TREATING VIRAL INFECTIONS

(71) Applicant: GODAVARI BIOREFINERIES LIMITED, Mumbai, Maharashtra (IN)

(72) Inventors: Maithili ATHAVALE, Mumbai, Maharashtra (IN); Sandip GAVADE, Mumbai, Maharashtra (IN); Prashant KHARKAR, Mumbai, Maharashtra (IN); Sangeeta SRIVASTAVA, Mumbai, Maharashtra (IN)

(21) Appl. No.: 17/998,485

(22) PCT Filed: May 10, 2021
(86) PCT No.: PCT/IN2021/050451

§ 371 Date: Nov. 10, 2022

(30) Foreign Application Priority Data
May 11, 2020 (IN) 202021019866

(51) Int. Cl. A61K 31/7048; A61K 31/4025; A61K 31/36; A61K 31/496; A61K 31/5377; A61K 31/5375; A61P 31/14

(52) U.S. Cl. CPC A61K 31/7048; A61K 31/4025; A61K 31/36; A61K 31/496; A61K 31/5377; A61K 31/5375; A61P 31/14

(57) ABSTRACT

The invention relates to use of a compound of Formula I for inhibition of V-ATPase activity in a cell and a method of treating viral infections using the compounds of Formula I.

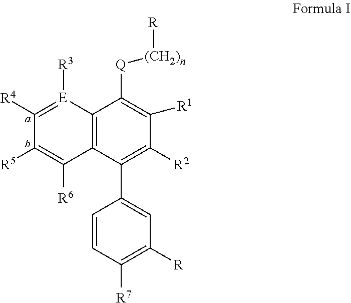

### FIELD OF THE INVENTION

The present invention relates to the use of compounds for inhibiting V-ATPase activity in a cell, particularly as inhibitors of V-ATPase activity of virus such as SARS-CoV-2 virus or influenza virus for treating viral infections.

### BACKGROUND OF THE INVENTION

The SARS-CoV-2 virus and the disease COVID-19 has created havoc globally without any cultural, demographical, technological and religious distinction.

In developing the solutions for the treatment of COVID 19, logical solutions such as Computational and Experimental Drug Repurposing or validating second medical uses come handy. Existing drugs can provide a starting point and significantly shorten the clinical development timelines. The literature is full of drug repurposing campaigns for targeting various events in the viral life cycle, starting with viral entry till the release of mature viral particles. The use of hydroxychloroquine, chloroquine and other antiviral drugs as a possible treatment option for COVID-19 is a testimony of the potential this approach holds.

One such promising target, is vacuolar ATPase (V-ATPase), as inferred from the antiviral efficacy of V-ATPase inhibitors such as bafilomycin. The V-ATPase has been proposed to be a promising target for intercepting virus entry into the host cells. V-ATPases are ubiquitous proton pumps located in the endomembrane system, i.e., endoplasmic reticulum (ER), golgi bodies, etc., of all eukaryotic cells. Viruses such as influenza viruses, flaviviruses, vaccinia viruses, bornaviruses, rhabdoviruses, and coronaviruses utilize V-ATPase-mediated endosomal acidification as a crucial cellular process for entry into the host cells.

V-ATPase inhibitors can be a potential intervention for viral entry, with much less susceptibility to development of drug resistance, since V-ATPase is a host protein. Several V-ATPase inhibitors have been investigated for their antiviral potential, such as bafilomycin (first discovered and the most notable example). Despite of their potent antiviral efficacy, toxicity was the main hurdle in their clinical application. In addition, the poor aqueous solubility of V-ATPase inhibitor is a concern for drug delivery modalities.

In view of the crucial role of V-ATPase in coronavirus, for example SARS-CoV-2 infection and the utility of its inhibitors in combating COVID-19, a need to develop potent, efficacious, V-ATPase inhibitors as potential therapeutics for targeting coronavirus infections is required.

### SUMMARY OF THE INVENTION

In an aspect, the present invention relates to use of a compound of Formula I for inhibiting V-ATPase activity in a cell,

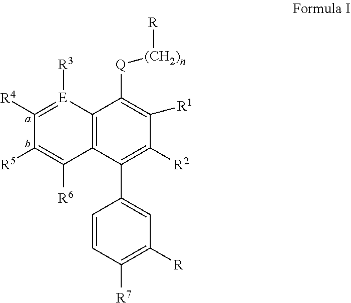

wherein,
E is selected from C, N;
Q is O, —NH;
n is 0-6;
R¹ and R² is selected from —H, —C(O)O-alkyl such as —C(O)OC₂H₅, or R¹ and R² together form a substituted or unsubstituted 5- or 6- membered ring such as lactone;
R³, R⁴, R⁵and R⁶ each independently is selected from —H, —OH, alkoxy;
R⁷ and R⁸ is selected from —H, —OH, alkoxy, —X where X can be F, Cl, Br or R⁷ and R⁸ together form a 5 membered ring containing one or more heteroatoms such as O;
a and b denote the presence or absence of double bond at the respective positions;
R¹¹ and R¹² each independently is selected from —H, or and R¹² can be substituted or unsubstituted 5- or 6- membered ring such as lactone, —nC(O)O-alkyl;
R is selected from substituted or unsubstituted phenyl ring,

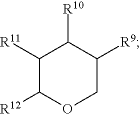

wherein R⁹, R¹⁰, R¹¹ and R¹² each independently is selected from —H, —OH, alkoxy,

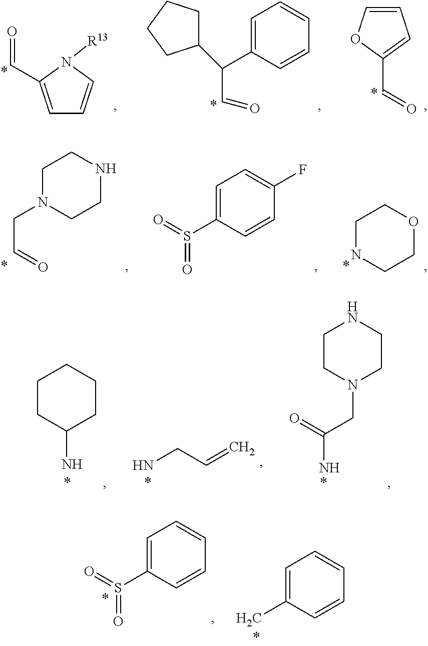

wherein R¹³ is H, alkyl; and
* represents linkage with Q or —CH₂ group.

In another aspect, the invention relates to use of a compound of Formula E for inhibition of V-ATPase activity in a cell,

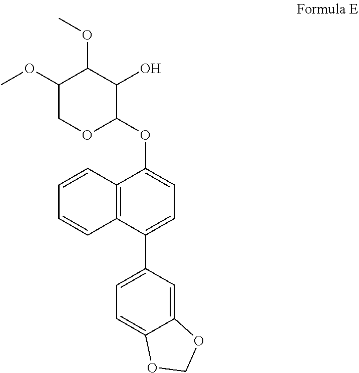 [SMILES: COC1COC(Oc2ccc(-c3ccc4c(c3)OCO4)c3ccccc23)C(O)C1OC]

### BRIEF DESCRIPTION OF DRAWINGS

FIG. 1: illustrates schematic representation of the ELISA assay.

FIG. 2: illustrates standard curve for V-ATPase in a concentration range of 31.25 pg/ml to 2000 pg/ml.

FIG. 3: illustrates the dose response curve of Remdesivir.

FIG. 4: illustrates the dose response curve of compound of Formula E of the present invention.

FIG. 5: illustrates effect of administration of compound of Formula E on SARS-CoV-2 infection in hamster, FIG. 5 (A) shows percent change in body mass of the hamsters from the day of challenge till 4th day post infection; FIG. 5 (B) shows image of whole lung as excised from the euthanized animals showing inflammation and pneumonitis; FIG. 5 (C) shows images of spleen showing splenomegaly condition.

FIG. 6: illustrates anti-viral and immunomodulatory efficacy of administration of compound of Formula E on SARS-CoV-2 infection in hamster, FIG. 6 (A) shows bar graph showing relative lung viral load in different groups on the 4th day post infection; FIG. 6 (B) shows bar graphs showing relative mRNA expression of cytokines in the spleen of different experimental groups. Each bar represent mean ±SEM.

FIG. 7: illustrates effect of administration of compound of Formula E on the lung pathology of SARS-CoV-2 infected hamster.

### DETAILED DESCRIPTION OF THE INVENTION

The present invention relates to use of a compound of Formula I for inhibiting V-ATPase activity in a cell,

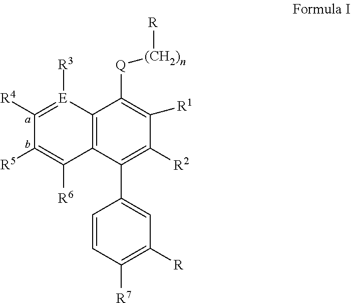

wherein,
E is selected from C, N;
Q is O, —NH;
n is 0-6;
R¹ and R² is selected from —H, —C(O)O-alkyl such as —C(O)OC₂H₅, or R¹ and R² together form a substituted or unsubstituted 5- or 6- membered ring such as lactone;
R³, R⁴, R⁵and R⁶ each independently is selected from —H, —OH, alkoxy;
R⁷ and R⁸ is selected from —H, —OH, alkoxy, —X where X can be F, Cl, Br or R⁷ and R⁸ together form a 5 membered ring containing one or more heteroatoms such as O;
a and b denote the presence or absence of double bond at the respective positions.
R¹¹ and R¹² each independently is selected from —H, or R¹¹ and R¹² can be substituted or unsubstituted 5- or 6- membered ring such as lactone, —C(O)O-alkyl;

R is selected from substituted or unsubstituted phenyl ring,

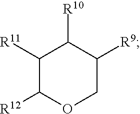

wherein R⁹, R¹⁰, R¹¹ and R¹² each independently is selected from —H, —OH, alkoxy,

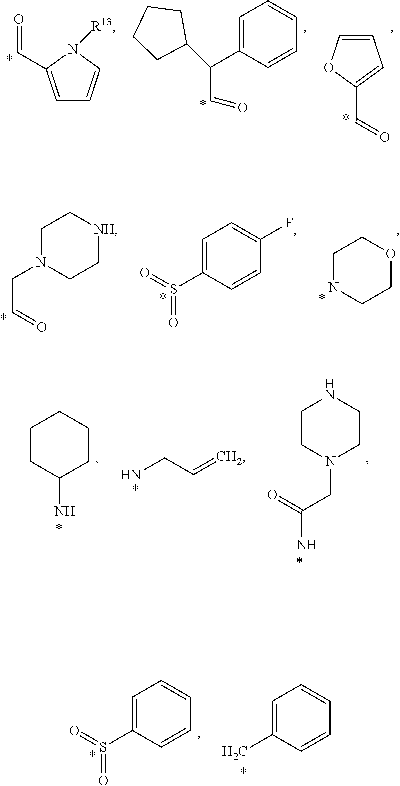

wherein R¹³ is H, alkyl; and
* represents linkage with Q or —CH₂ group.

In an embodiment, a and b in Formula I denote the absence of double bond at the respective positions. For example, when E is —N and R³ is present, the double bond at the respective position will be absent or when E is —N and R³ is absent, the double bond at the respective position will be present.

In an embodiment, the invention relates to the use of a compound of Formula II for inhibiting V-ATPase activity in a cell,

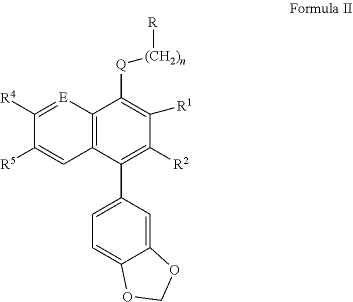

In an embodiment, the invention relates to the use of a compound of Formula III for inhibiting V-ATPase activity in a cell,

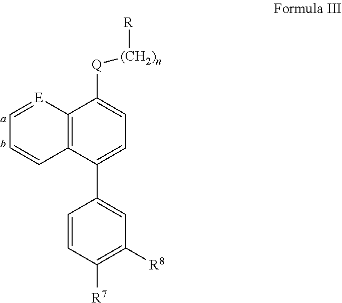

The groups are same as defined above in Formula I.

In an embodiment, the present invention relates to the use of the compounds shown in Table 1 for inhibiting V-ATPase in a cell. The compounds act as anti-viral agents in the treatment of SARS-CoV-2 or influenza infections.

TABLE 1

| Formula | IUPAC Name | Structure |
|---|---|---|
| A | 9-(benzo[d][1,3]dioxol-5-yl)- 6,7-dimethoxy-4-(tetrahydro-3- hydroxy-4,5-dimethoxy-2H- pyran-2-yloxy)naphtho[2,3- c]furan-1(3H)-one | 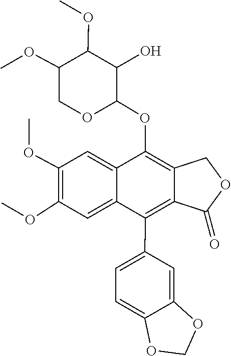 [SMILES: COc1cc2c(OC3OCC(OC)C(OC)C3O)c3c(c(-c4ccc5c(c4)OCO5)c2cc1OC)C(=O)OC3] |
| B | 9-(benzo[d][1,3]dioxol-5-yl)- 1,3-dihydro-6,7-dimethoxy-1- oxonaphtho[2,3-c]furan-4-yl 1- methyl-1H-pyrrole-2- carboxylate | 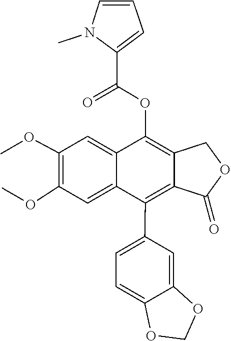 [SMILES: COc1cc2c(OC(=O)c3cccn3C)c3c(c(-c4ccc5c(c4)OCO5)c2cc1OC)C(=O)OC3] |
| C | 9-(benzo[d][1,3]dioxol-5-yl)- 1,3-dihydro-6,7-dimethoxy-1- oxonaphtho[2,3-c]furan-4-yl 2- cyclopentyl-2-phenylacetate | 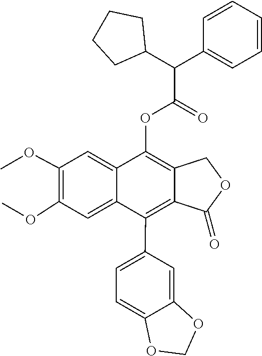 [SMILES: COc1cc2c(OC(=O)C(c3ccccc3)C3CCCC3)c3c(c(-c4ccc5c(c4)OCO5)c2cc1OC)C(=O)OC3] |
| D | diethyl 1-(benzo[d][1,3]dioxol- 5-yl)-6,7-dimethoxy-4- (tetrahydro-3-hydroxy-4,5- dimethoxy-2H-pyran-2- yloxy)naphthalene-2,3- dicarboxylate | 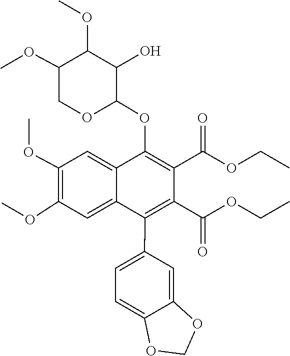 [SMILES: CCOC(=O)c1c(C(=O)OCC)c(-c2ccc3c(c2)OCO3)c2cc(OC)c(OC)cc2c1OC1OCC(OC)C(OC)C1O] |
| E | 2-(1-(benzo[d][1,3]dioxol-5- yl)naphthalen-4-yloxy)- tetrahydro-4,5-dimethoxy-2H- pyran-3-ol | 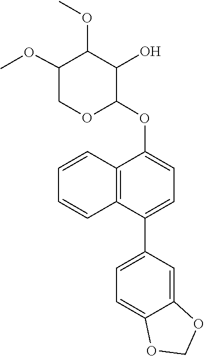 [SMILES: COC1COC(Oc2ccc(-c3ccc4c(c3)OCO4)c3ccccc23)C(O)C1OC] |
| F | (3R,4S,5R)-2-(1- (benzo[d][1,3]dioxol-5- yl)naphthalen-4-yloxy)- tetrahydro-2H-pyran-3,4,5-triol | 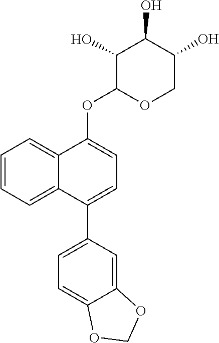 [SMILES: O[C@H]1[C@H](O)COC(Oc2ccc(-c3ccc4c(c3)OCO4)c3ccccc23)[C@@H]1O] |
| G | N-(1-(benzo[d][1,3]dioxol-5- yl)naphthalen-4-yl)furan-2- carboxamide | 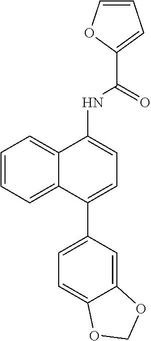 [SMILES: O=C(Nc1ccc(-c2ccc3c(c2)OCO3)c2ccccc12)c1ccco1] |
| H | 5-(benzo[d][1,3]dioxol-5-yl)-8- (benzyloxy)quinoline | 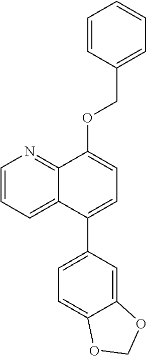 [SMILES: c1ccc(COc2ccc(-c3ccc4c(c3)OCO4)c3cccnc23)cc1] |
| I | N-(1-(benzo[d][1,3]dioxol-5- yl)naphthalen-4- yl)benzenesulphonamide | 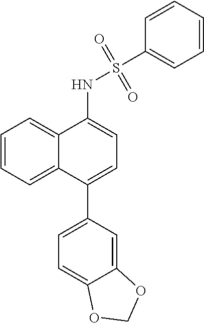 [SMILES: O=S(=O)(Nc1ccc(-c2ccc3c(c2)OCO3)c2ccccc12)c1ccccc1] |
| J | N-(1-(benzo[d][1,3]dioxol-5- yl)naphthalen-4-yl)-4- fluorobenzenesulphonamide | 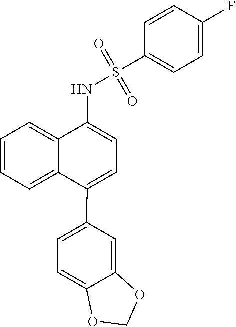 [SMILES: O=S(=O)(Nc1ccc(-c2ccc3c(c2)OCO3)c2ccccc12)c1ccc(F)cc1] |
| K | N-(1-(benzo[d][1,3]dioxol-5- yl)naphthalen-4-yl)-2- (piperazin-1-yl)acetamide | 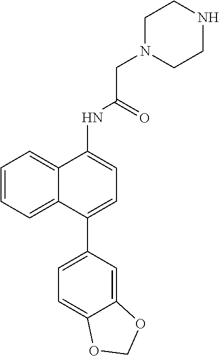 [SMILES: O=C(CN1CCNCC1)Nc1ccc(-c2ccc3c(c2)OCO3)c2ccccc12] |
| L | 4-(4-morpholinobutoxy)-9- (benzo[d][1,3]dioxol-5-yl)-6,7- dimethoxynaphtho[2,3-c]furan- 1(3H)-one | 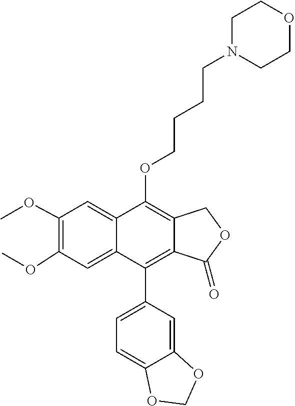 [SMILES: COc1cc2c(OCCCCN3CCOCC3)c3c(c(-c4ccc5c(c4)OCO5)c2cc1OC)C(=O)OC3] |
| M | 4-(4-(1-(benzo[d][1,3]dioxol-5- yl)naphthalen-4- yloxy)butyl)morpholine | 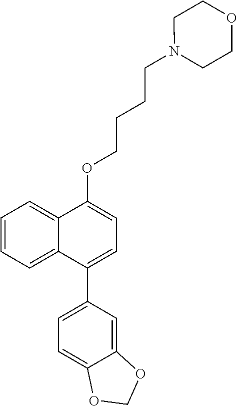 [SMILES: c1ccc2c(-c3ccc4c(c3)OCO4)ccc(OCCCCN3CCOCC3)c2c1] |
| N | N-(4-(1-(benzo[d][1,3]dioxol-5- yl)naphthalen-4- yloxy)butyl)cyclohexanamine | 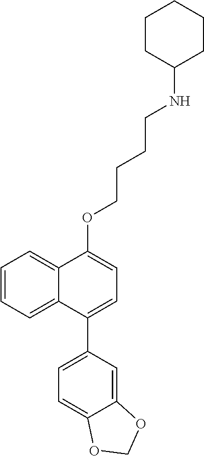 [SMILES: c1ccc2c(-c3ccc4c(c3)OCO4)ccc(OCCCCNC3CCCCC3)c2c1] |
| O | 4-(2-(1-(benzo[d][1,3]dioxol-5- yl)naphthalen-4- yloxy)ethyl)morpholine | 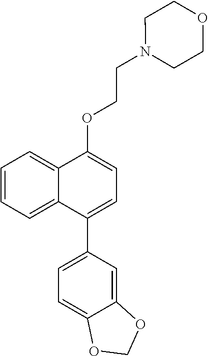 [SMILES: c1ccc2c(-c3ccc4c(c3)OCO4)ccc(OCCN3CCOCC3)c2c1] |
| P | 4-(2-(1-(3- methoxyphenyl)naphthalen-4- yloxy)ethyl)morpholine | 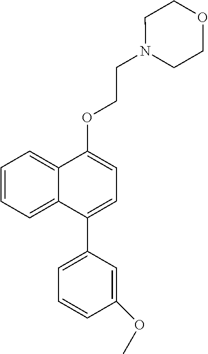 [SMILES: COc1cccc(-c2ccc(OCCN3CCOCC3)c3ccccc23)c1] |
| Q | N-(6-(5-(benzo[d][1,3]dioxol-5- yl)-1,2,3,4- tetrahydronaphthalen-8- yloxy)hexyl)cyclohexanamine | 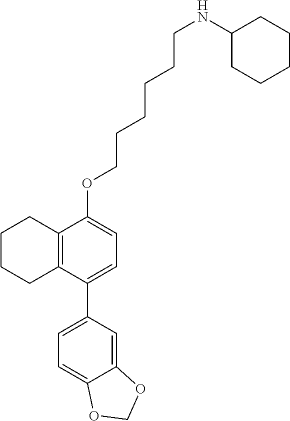 [SMILES: c1cc2c(cc1-c1ccc(OCCCCCCNC3CCCCC3)c3c1CCCC3)OCO2] |
| R | 6-(5-(benzo[d][1,3]dioxol-5-yl)- 1,2,3,4-tetrahydronaphthalen-8- yloxy)-N-allylhexan-1-amine | 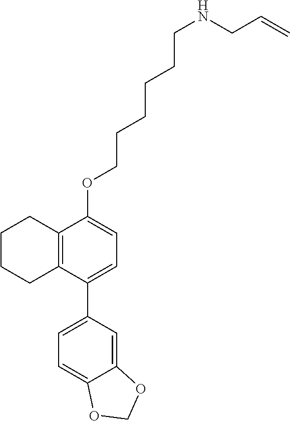 |
| S | 8-(2-morpholinoethoxy)-5- (benzo[d][1,3]dioxol-5- yl)quinoline | 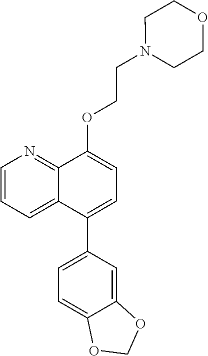 [SMILES: c1cnc2c(OCCN3CCOCC3)ccc(-c3ccc4c(c3)OCO4)c2c1] |
| T | N-(2-(1-(benzo[d][1,3]dioxol-5- yl)naphthalen-4-yloxy)ethyl)-2- (piperazin-1-yl)acetamide | 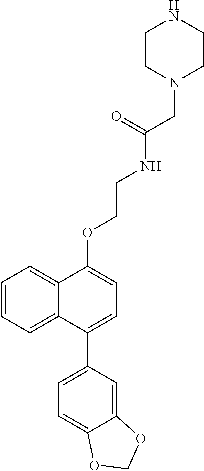 [SMILES: O=C(CN1CCNCC1)NCCOc1ccc(-c2ccc3c(c2)OCO3)c2ccccc12] |
| U | 4-(2-(1-(3,4- dimethoxyphenyl)naphthalen-4- yloxy)ethyl)morpholine | 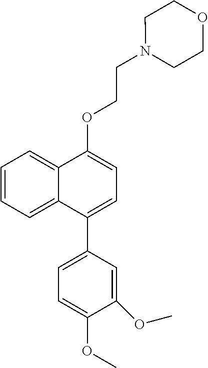 [SMILES: COc1ccc(-c2ccc(OCCN3CCOCC3)c3ccccc23)cc1OC] |

In another aspect, the invention relates to use of a compound of Formula E for inhibition of V-ATPase activity in a cell. Compound of Formula E is effective in the treatment of SARS-CoV-2 and influenza infections.

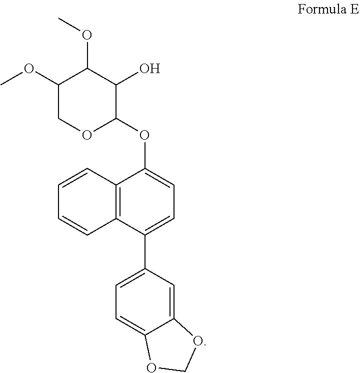 [SMILES: COC1COC(Oc2ccc(-c3ccc4c(c3)OCO4)c3ccccc23)C(O)C1OC]

The compound of Formula E has a molecular weight of around 450 and a partition co-efficient (log P) value of near to 4. The compound is soluble in solvents like dimethyl sulfoxide (DMSO), methanol, ethyl acetate.

The use of the compounds of Formula I to III and Formula A to U can be as prodrugs, metabolites, pharmaceutically acceptable salts, solvates or polymorphs thereof.

The use of the compounds of Formula Ito III and Formula A to U is for inhibition of a virus. V-ATPase activity in a cell infected with a virus is inhibited. It is understood that inhibition of V-ATPase activity in a cell inhibits the virus.

The use of the compounds of Formula Ito III and Formula A to U is for inhibition of virus, such as SARS-CoV-2 virus or influenza virus.

The use of compound of Formula E is for inhibition of SARS-CoV-2 virus or influenza virus.

The invention also relates to a method of inhibiting V-ATPase activity in a cell by contacting the cell with at least one or more compounds of Formula I to III and Formula A to U.

The invention also relates to a method of inhibiting a virus by contacting the virus infected cell with at least one or more compounds of Formula Ito III and Formula A to U, preferably, the method of inhibiting is for SARS-CoV-2 virus or influenza virus.

The method of inhibiting V-ATPase activity in a cell comprises of contacting the cell or virus infected cell with the compound of Formula E.

The invention also relates to a method of treatment of viral infections, by administering a therapeutically effective amount of one or more of the compounds of Formula Ito III and Formula A to U to inhibit V-ATPase activity in a cell. The method of treatment is preferably for treatment of SARS-CoV-2 virus or influenza virus infection.

In an embodiment, the method of treatment comprises administering therapeutically effect amount of compounds of Formula Ito III and Formula A to U in combination with at least one additional other compound having V-ATPase inhibitory activity.

In another embodiment, the method of treatment comprises administering a compound of Formula E as one of the compounds.

The compounds of the present invention are highly effective for inhibition of V-ATPase activity in a cell. The compounds have shown to demonstrate good anti-viral effect results both in in-vitro and in-vivo studies without having a cytotoxic effect on the normal human lymphocytes. This indicates that the compounds can be effectively and safely used for treating viral infections, particularly SARS-CoV-2 infections.

In an embodiment, the compounds encompassed by the present invention are used in preparation of medicament for use in the treatment of viral infections such as SARS-CoV-2 or influenza infections by inhibiting V-ATPase activity in a cell.

Few compounds encompassed by the scope of the present invention are disclosed herewith. It must be noted that the disclosed compounds do not limit the scope of the present invention.

### EXAMPLES

The compounds of the present invention are prepared by known methods. The method of preparation of the compounds as disclosed in WO2018193476 and WO2020129082 is incorporated herein by reference.

### EXAMPLE 1

### Determination of V-ATPase Inhibitory Activity in a Cell

The Vacuolar ATPase (V-ATPase) is a proton pump responsible for controlling the intracellular and extracellular pH of cells. The structure of V-ATPase has been highly conserved among all eukaryotic cells and is involved in diverse functions across species. V-ATPase is best known for its acidification of endosomes and lysosomes and is also important for luminal acidification of specialized cells.

Among host factors that can be targeted for antiviral treatments, V-ATPases are a promising targets for intercepting virus entry into host cells. Among viral threats such as influenza viruses, flaviviruses, vaccinia viruses, bornaviruses, rhabdoviruses and Coronaviruses. V-ATPase mediated endosomal acidification may thus pave ways to new antiviral treatments with broad applicability and low susceptibility to drug-resistant mutation.

An ELISA test was performed using Human V-Type Proton ATPase ELISA kit (My BioSource Catlog no: MBS911862) to determine the V-ATPase inhibitory effect of the compounds of the present invention.

Procedure: 1.0 million cells of MDAMB231 (cell line rich in V-ATPase) were plated on 60 mm plates for 24 hours at 37° C. and 5% CO₂. The cells were treated with the test compounds (Formula A to U) at a concentration of 100 nM (0.1 μM)/10,000 cells and incubated for 48 hours. After incubation the supernatant was discarded, the cells were washed with D.P.B.S. (Dulbecco's phosphate-buffered saline), scraped and stored at −80° C. for 48 hours. The cells were thawed, vortexed, homogenized for 7 minutes, followed by centrifugation for 3 minutes at 3000 R.P.M. The supernatant was taken for V-ATPase Assay.

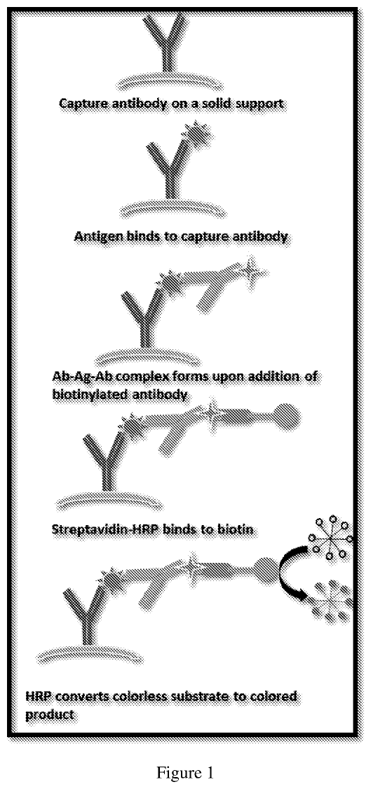

The assay employed the quantitative sandwich enzyme immunoassay technique as shown in FIG. 1.

Standards and samples were pipetted into the wells. Antibody specific for V-ATPase was pre-coated onto a microplate. Any V-ATPase present in the standard /sample would get bound by the immobilized antibody. After the incubation period any unbound substances were removed by washing and a biotin-conjugated antibody specific for V-ATPase was added to the wells and plates were re-incubated at 37° C. and 5% CO₂. After washing, avidin conjugated Horseradish Peroxidase (HRP) was added to the wells. Following a wash to remove any unbound avidin-enzyme reagent, a substrate solution was added to the wells and colour developed was in proportion to the amount of V-ATPase bound in the initial step. The colour development was stopped, and the optical density was measured at 450 nm spectrophotometrically. The concentration of V-ATPase was then calculated by substituting the optical density in the regression equation obtained from the standards. Percent (%) reduction in V-ATPase was calculated by subtracting the amount of V-ATPase obtained from the untreated/conc. of V-ATPase in untreated X100.

### Results

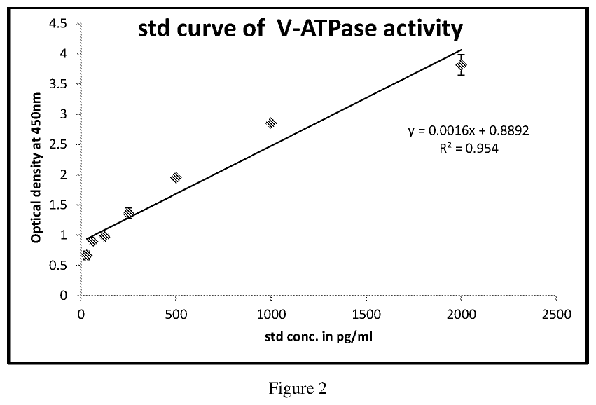

A standard curve for V-ATPase concentration in range of 31.25 pg/ml to 2000 pg/ml was plotted as shown in FIG. 2, the plot exhibited a good linearity (R²=0.954).

Table 2 shows the result of V-ATPase assay of the compounds of the present invention.

TABLE 2

| Sample | reduction of V-ATPase (%) |
|---|---|
| *Untreated MDAMB231 cells (100% | 0 |
| concentration of V-ATPase) | |
| Compound of Formula A | 13.31 |
| Compound of Formula B | 15.33 |
| Compound of Formula C | 14.69 |
| Compound of Formula D | 18.2 |
| Compound of Formula E | 15.6 |
| Compound of Formula F | 61.0 |
| Compound of Formula G | 62.37 |
| Compound of Formula H | 61.34 |
| Compound of Formula I | 20.56 |
| Compound of Formula J | 60.96 |
| Compound of Formula K | 28.05 |
| Compound of Formula L | 21.0 |
| Compound of Formula M | 76.16 |
| Compound of Formula N | 6.3 |
| Compound of Formula O | 20.26 |
| Compound of Formula P | 18.27 |
| Compound of Formula Q | 77.42 |
| Compound of Formula R | 16.2 |
| Compound of Formula S | 75.5 |
| Compound of Formula T | 81.9 |
| Compound of Formula U | 16.57 |

*MDAMB231 cell lines are used in the present study, as these are rich in V-ATPase

### Conclusion

The results indicate that the compounds of Formula A to U resulted in reduction of V-ATPase activity in the cells.

### EXAMPLE 2

Toxicity studies of compounds of the present invention on normal cells The toxicity study was performed on Human Peripheral Blood Lymphocytes obtained differential centrifugation of defibrinated blood. MTT assay for compound of the present invention was performed to determine its toxicity. MTT assay is a simple and sensitive assay where, metabolic reducing activity of the cells is measured. It was carried out in the following manner.

A required volume of cell suspension was prepared as per the cell plating efficiency. 200 μl of prepared cell suspension was added in labelled 96 well plates and the plates were placed in an incubator for 18-24 hours at 37° C. and 5% CO₂. Then, 2 μl of respective compound dilution was added and the plates were placed in the incubator for 48 hours at 37° C. and 5% CO₂. The suspension was aspirated from the plates and 100 μl of working MTT solution (0.5 mg/ml MTT prepared from 5 mg/ml MTT stock in 1× complete media) was added. The plates were incubated in the incubator for 4 hours at 37° C. and 5% CO₂. The plates were spinned down, supernatant removed, 200 μl DMSO was added, mixed gently and placed in the incubator for 10 minutes at 37° C. and 5% CO₂. The absorbance at 570 nm was read and percentage viability and IC₅₀ value of the compounds was calculated using regression analysis.

Table 3 shows the results of the MTT assay conducted for the compounds of the present invention.

TABLE 3

| Sample | IC₅₀ (μM) |
|---|---|
| Compound of Formula B | No activity (Nontoxic) |
| Compound of Formula C | No activity (Nontoxic) |
| Compound of Formula D | No activity (Nontoxic) |
| Compound of Formula E | No activity (Nontoxic) |
| Compound of Formula I | No activity (Nontoxic) |
| Compound of Formula L | No activity (Nontoxic) |
| Compound of Formula O | No activity (Nontoxic) |
| Compound of Formula P | No activity (Nontoxic) |
| Compound of Formula U | No activity (Nontoxic) |

### Conclusion

From the above results it was seen that compounds of Formula B to E, I, L, O, P, U did not exhibit any toxicity towards normal human lymphocytes up to 100 μM. This indicates that the compounds of the present invention did not exhibit any toxicity on normal peripheral blood lymphocytes.

### Other Safety Studies

Various pre-clinical GLP (Good Laboratory Practice) and non-GLP studies were performed with the compound of Formula E. These included 7-day and 28- days studies on rats and dogs, acute toxicity in rats, mice and dogs. The effect of the compound of Formula E on respiratory function, nervous system and cardiovascular system, bacterial mutation, metabolism, CaCo2 permeability, protein binding and cytochrome effect was also studied. These studies showed that the compound of Formula E was non-toxic, non-mutagenic, non-clastogenic, and showed no effect on the respiratory, nervous and cardiovascular system. Further, the compound of Formula E did not show any toxic or adverse effect even at a higher dose of 2000 mg/kg in rats, mice and dogs. This indicates that the compound of Formula E was safe for administration.

### Example 3

### In-Vitro Anti SARS-CoV-2 Activity of the Compounds of the Present Invention

### Neutral Red (Cytopathic Effect/Toxicity Assay)

### Procedure:

### Reduction of Virus-Induced Cytopathic Effect (Primary CPE Assay)

Confluent or near-confluent cell culture monolayers of Vero 76 cells were prepared in 96-well disposable microplates the day before testing. Cells were maintained in MEM (Minimum Essential Medium Eagle) supplemented with 5% FBS (Fetal Bovine Serum). For antiviral assays the same medium was used but with FBS reduced to 2% and supplemented with 50-μg/ml gentamicin.

Compounds were dissolved in DMSO, saline or the diluent. Less soluble compounds were vortexed, heated, and sonicated, and if they still did not go into solution were tested as colloidal suspensions. The test compound was prepared at four serial log₁₀ concentrations, usually 0.1, 1.0, 10, and 100 μg/ml or μM. Lower concentrations were used when insufficient compound was supplied. Five microwells were used per dilution: three for infected cultures and two for uninfected toxicity cultures. Controls for the experiment consisted of six microwells that were infected and not treated (virus controls) and six that were untreated and uninfected (cell controls) on every plate. A known active drug was tested in parallel as a positive control drug using the same method as was applied for test compounds. The positive control was tested with every test run.

Growth media was removed from the cells and the test compound was applied in 0.1 ml volume to wells at 2× concentration. Virus, normally at ˜60 CCID₅₀ (50% cell culture infectious dose) in 0.1 ml volume is added to the wells designated for virus infection. Medium devoid of virus was placed in toxicity control wells and cell control wells. Plates were incubated at 37° C. with 5% CO₂ until marked CPE (>80% CPE for most virus strains) was observed in virus control wells. The plates were then stained with 0.011% neutral red for approximately two hours at 37° C. in a 5% CO₂ incubator. The neutral red medium was removed by complete aspiration, and the cells may be rinsed 1× with phosphate buffered solution (PBS) to remove residual dye. The PBS was completely removed, and the incorporated neutral red was eluted with 50% Sorensen's citrate buffer/50% ethanol for at least 30 minutes. Neutral red dye penetrates into living cells, thus, the more intense the red color, the larger the number of viable cells present in the wells. The dye content in each well was quantified using a spectrophotometer at 540 nm wavelength. The dye content in each set of wells was converted to a percentage of dye present in untreated control wells using a Microsoft Excel computer-based spreadsheet and normalized based on the virus control. The 50% effective (EC₅₀, virus-inhibitory) concentrations and 50% cytotoxic (CC₅₀, cell-inhibitory) concentrations are then calculated by regression analysis. The quotient of CC₅₀ divided by EC₅₀ gives the selectivity index (SI) value.

Table 3 shows the results of Neutral Red (Cytopathic effect/Toxicity Assay).

TABLE 3

| Compound | EC₅₀ (μg/ml) | CC₅₀ (μg/ml) | SI₅₀ |
|---|---|---|---|
| Control | 0.34 | >100 | >290 |
| Formula A | >0.1 | <0.1 | 0 |
| Formula E | 0.35 | 2.5 | 7.1 |
| Formula F | 2.9 | 32 | 11 |
| Formula D | 32 | 40 | 1.3 |
| Formula L | >0.34 | 0.34 | 0 |

For Neutral Red Assay, the compound of Formula E had EC₅₀ , CC₅₀ and SI₅₀ value of 0.35 μg/ml, 2.5 μg/ml and 7.1, respectively. The values indicate that the compounds, particularly compound of Formula E inhibits virus replication.

### Conclusion

As the compound of Formula E shows EC₅₀-0.35 and SI₅₀- 7.1 and compound of Formula F shows EC₅₀-2.9 and SI₅₀-11, they are considered to have good anti-viral activity.

### Example 4

### In-Vitro Anti SARS-CoV-2 Activity of Compound of Formula E (Assay Method-IC₅₀ Estimation)

### Procedure:

The assay was done in a 96-well plate format in 3 wells for each sample. 1×10e4 VeroE6 cells were plated per well and incubated at 37° C. overnight for the monolayer formation. Next day, cells were incubated with the test substance (TS) at the 7-point concentration. For Remdesivir, the following concentrations were used: 10 μM, 3 μM, 1 μM, 0.3 μM, 0.1 μM, 0.03 μM and 0.01 μM. For the compound of Formula E, 10 μM stock solution was serially diluted in DMSO (2-fold dilution). From each dilution, 2-microliter was used for the screening assay. The 7-point concentrations of the compound of Formula E were 10 μg, 3 μg, 1 μg, 0.3 μg, 0.1 μg, 0.03 μg and 0.01 μg. The control cells were incubated with 0.5% DMSO only. The cells were infected with SARS-CoV-2 at a MOI of 0.01. 48 hours later, viral RNA was extracted from 100 μl culture supernatant and subjected to qRT-PCR (in duplicates) where Ct values for N and E gene sequence were determined. Inhibition of virus replication was determined based on the fold change in the Ct value in TS-treated cells compared to the control. IC₅₀ values were determined using AAT Bioquest IC₅₀ calculator as shown in Table 4.

TABLE 4

IC₅₀ value (μM)

| Compound | E gene | N gene |
|---|---|---|
| Remdesivir | 0.15 | 0.11 |
| Compound of Formula E | 0.5713 | 0.5857 |

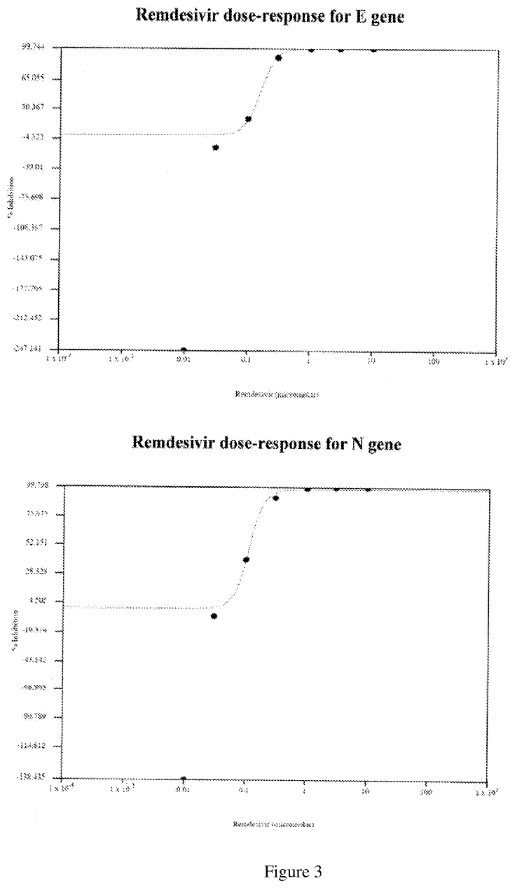

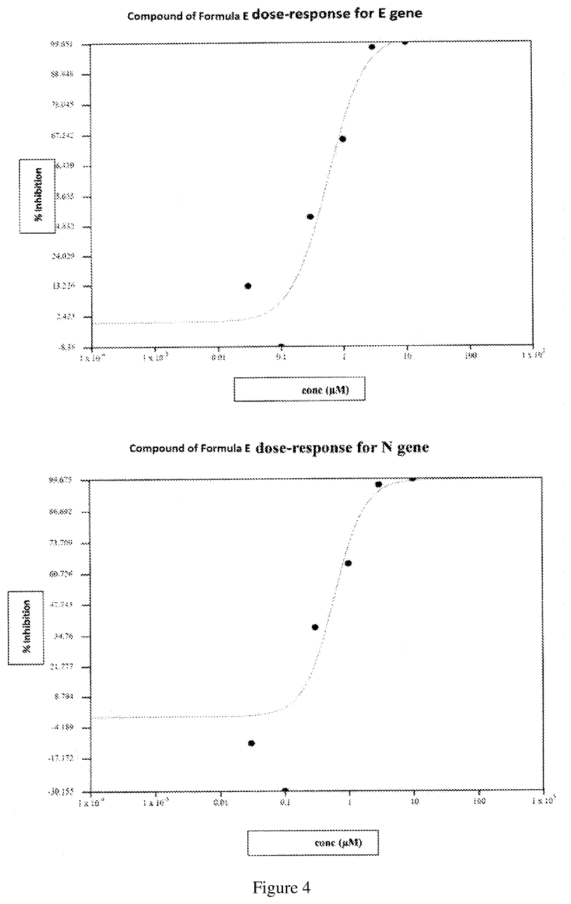

FIG. 3 shows the dose response curve for Remdesivir. FIG. 4 shows the dose response curve for compound of Formula E.

### Conclusion

The results indicate that the compound of Formula E exhibited good anti-SARS-CoV-2 activity (IC₅₀<10 μM) in in-vitro cytotoxicity assay.

### Example 5

### In-Vitro Anti SARS-CoV-2 Activity of Compound of Formula E

### Assay Method—Cytotoxicity

### Procedure

The assay was done in a 96 well plate format in 3 wells for each sample. 1×10e4 Vero E6 cells were plated per well and incubated at 37° C. overnight for monolayer formation. Next day, cells were incubated with the test substance (TS) at the indicated concentration with final DMSO concentration being 0.5%. The Control cells were incubated with 0.5% DMSO only. 24 and 48 hours later, cells were stained with Hoechst 33342 and Sytox orange dye. Images were taken at 10×, 16 images per well, which covers 90% of the well area using ImageXpress Microconfocal (Molecular Devices). Hoechst 33342 nucleic acid stain is a popular cell-permanent nuclear counterstain that emits blue fluorescence when bound to dsDNA. It stains all the live and dead cells. Sytox orange dye stains the nucleic acid in the cells with compromised membranes. This stain is an indicator of cell death. First, the software will count total number of cells in the Hoechst image. In the Sytox image, it will count, among Hoechst positive cells, how many cells are positive for Sytox.

### Antiviral Screening

### Procedure:

The assay was done in a 96-well plate format in 3 wells for each sample. 1×10e4 VeroE6 cells were plated per well and incubated at 37° C. overnight for the monolayer formation. Next day, cells were incubated with the test substance (TS) at the indicated concentration with final DMSO concentration being 0.5%. The Control cells were incubated with 0.5% DMSO only. The cells were infected with SARS-CoV-2 at a MOI of 0.01. 24 and 48 hours later, viral RNA was extracted from 100 μl A culture supernatant and subjected to qRT-PCR (in duplicates) where Ct values for N and E gene sequence were determined. Inhibition of virus replication was determined based on the fold change in the Ct value in TS-treated cells compared to the control. Remdesivir was used as a positive control for viral inhibition.

### Results

Table 5 shows the results for cytotoxicity and antiviral activity of compound of Formula E.

TABLE 5

| | | % Cell | | % inhibition of virus replication | | | |
|---|---|---|---|---|---|---|---|
| | | viability | | 24 hours post | | 48 hours post | |
| compound | Conc. | 24 | 48 | infection | | infection | |
| Name | (μM) | hours | hours | E | N | E | N |
| Remdesivir | 10 | 99.23 | 94.37 | 82.38 | 80.35 | 99.8 | 99.89 |
| Compound | 10 | 99.60 | 77.81 | 75.39 | 81.3 | 99.5 | 99.7 |
| of Formula E | | | | | | | |

From the above results it can be seen that the compound of Formula E has slightly higher cell viability after 24 hours than Remdesivir. The percent inhibition viral replication after 24 hours and 48 hours of infection by the compound of Formula E was comparable to that of Remdesivir.

### Conclusion

As Remdesivir is effective against SARS-CoV-2 virus, compound of Formula E is also effective in inhibiting SARS-CoV-2 virus and can be effectively used in treatment of SARS-CoV-2 viral infection.

### Example 6

### In-Vivo Study for Anti SARS-CoV-2 Activity of Compound of Formula E

The in-vivo study was carried out on Syrian hamster infected with SARS-CoV-2 as described below.

### Methodology

Animals: 6-8 weeks old male golden Syrian hamsters were procured from CDRI (Central Drug Research Institute) and transported to small animal facility (SAF), THSTI (Translational Health Science and Technology Institute) and quarantined for 7 days prior to challenge study. During the pre-treatment regime the animals were housed at small animal facility (SAF) and then were transferred to the Animal biosafety level-3 (ABSL-3) institutional facility for SARS-CoV-2 challenge study. The animals were maintained for fewer than 12 h light and dark cycle and fed standard pellet diet and water ad libitum. All the experimental protocols involving immunization, booster dose and animal challenge were approved by institutional ethics committee IAEC (IAEC/THSTI/118) , IBS and RCGM. Virus culture and titration SARS-Related Coronavirus 2, Isolate USA-WA1/2020 virus was grown and titrated in Vero E6 cell line cultured in Dulbecco's Modified Eagle Medium (DMEM) complete media containing 4.5 g/L D-glucose, 100,000 U/L Penicillin-Streptomycin, 100 mg/L sodium pyruvate, 25 mM HEPES and 2% FBS. The stocks of virus were plaque purified at THSTI IDRF facility inside ABSL3 following institutional biosafety guidelines.

### Virus Culture and Titration

SARS-Related Coronavirus 2, Isolate USA-WA1/2020 virus was grown and titrated in Vero E6 cell line cultured in Dulbecco's Modified Eagle Medium (DMEM) complete media containing 4.5 g/L D-glucose, 100,000 U/L Penicillin-Streptomycin, 100 mg/L sodium pyruvate, 25 mM HEPES and 2% FBS. The stocks of virus were plaque purified at THSTI IDRF facility inside ABSL3 following institutional biosafety guidelines.

### SARS-CoV2 Infection in Golden Syrian Hamster and Dosing

33 male golden Syrian hamsters were randomly allotted to different groups (n=5), 1) challenge control (n=5), 2) Remdesivir control (n=5) and unchallenged control (n=3) were housed in separate cages. The pre-treatment group (δIII/p400) started receiving 400 mpk of the compound of Formula E (drug) through oral gavaging 2 days prior to the challenge. The other 3 groups viz δI/200, δII/800, δIV/400 received the compound of Formula E 200, 800 and 400 mpk post challenge through oral gavaging respectively for each day till the end point. All the animals, except unchallenged control, were challenged with 105 PFU of SARS-CoV-2 intranasal administration through catheter 50 μl in each nare under anaesthesia by using ketamine (150 mg/kg) and xylazine (10 mg/kg) intraperitoneal injection inside ABSL3 facility (Chan et al., 2020; Rizvi et. al., 2021, Sia et al., 2020). Unchallenged control group received mock PBS intranasally. All the experimental protocols involving the handling of virus culture and animal infection were approved by RCGM, institutional biosafety and IAEC animal ethics committee.

### Gross Clinical Parameters of SARS-CoV-2 Infection

All infected animals were euthanized on 4 days post infection at ABSL3. Changes in body weight, activity of the animals were observed on each day post challenge. Post sacrifice, lungs and spleen of the animals were excised and imaged for gross morphological changes. Right lower lobe of the lung was fixed in 10% neutral formalin solution and used for histological analysis. The complete left lobe of the lung was homogenized in 2 ml Trizol solution for viral load estimation. Spleen was homogenized in 2 ml of Trizol solution. The tissue samples in Trizol were stored immediately at −80° C. till further use. Blood of the animals were drawn through direct heart puncture and serum was isolated and stored at −80° C. till further use.

### Results

### Effect of Oral Administration of the Compound of Formula E on the Gross Clinical Parameter of SARS-CoV-2 Infected Hamsters.

To understand the protective efficacy of the compound of Formula E (according to the dosing regimen) against SARS-CoV-2 challenge in hamsters the gross clinical parameters associated with SARS-CoV-2 infection in hamsters were evaluated in infected groups receiving different doses of the compound of Formula E viz δI/200, δII/800, δIII/p400, δIV/400 against experimental control uninfected (UI), SARS-CoV-2 infected (I), hamsters receiving SC Remdesivir (R).

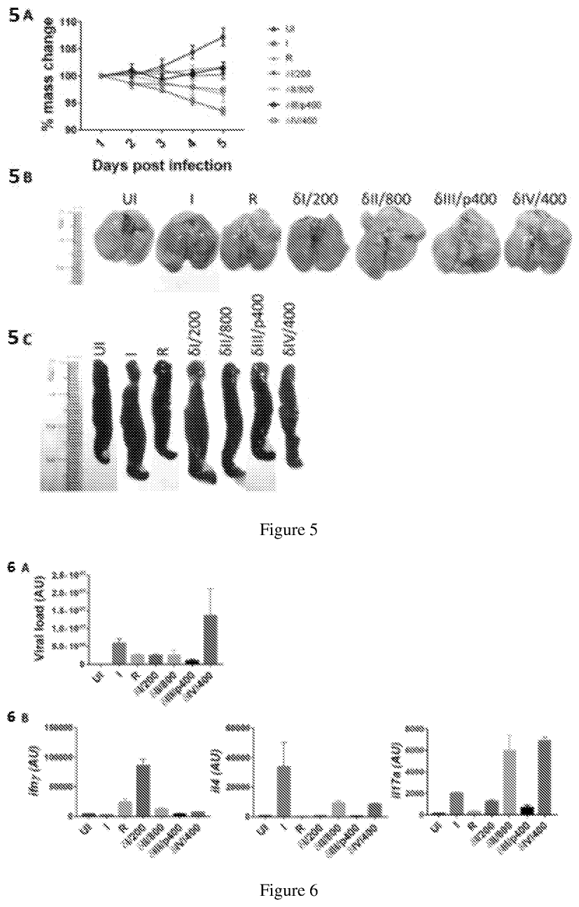

As shown in FIG. 5A, the results indicated that golden Syrian hamsters receiving δIII/p400 showed best protection against body mass loss and did not lose weight post infection. Further, δI/200 group showed second best recovery in terms of body mass loss while the other 2 groups showed a marginal protection against infected control but did show a gradual decrease in body mass post infection.

Consistently, lungs isolated from the euthanized animals on day 4 post challenge showed significantly lesser regions of pneumonitis and inflammation in the δIII/p400 followed by δII/800 group indicating a possibly less SARS-CoV2 associated lung damage in these two groups as compared to infected group (FIG. 5B). Splenomegaly, which is another clinical parameter of SARS-CoV2 associated pathology in hamster, showed significant mitigation in δIII/p400 group as compared to the infected control group, while there were little or no reduction in spleen size in other drug groups (FIG. 5C).

### Anti-Viral and Immunomodulatory Properties of the Compound of Formula E on SARS-CoV-2 Infected Hamsters.

To understand the anti-viral efficacy of the compound of Formula E relative viral load quantitation of SARS-CoV2 N2 gene from the excised lung of hamsters were carried out.

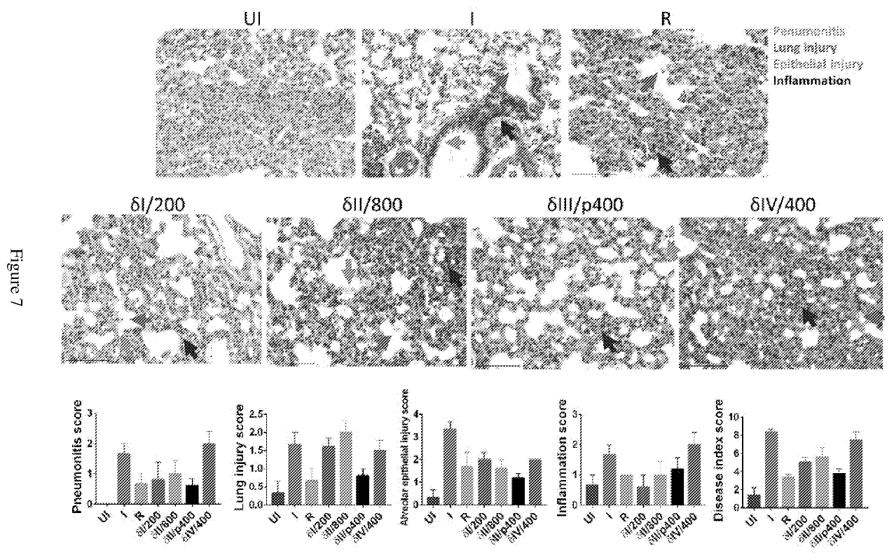

Results in FIG. 6A show significant reduction in relative lung viral load for δI/200, δII/800 and δIII/p400 group. The highest viral load reduction was seen in δIII/p400 showing 5 fold reduction in viral load as compared to the infected control group while other groups such as δI/200 and δII/800 showed around 2 to 2.5 fold reduction in lung viral load. Interestingly, there was no reduction in lung viral load in δIV/400 group and there was heterogeneity in the lung viral load in this group as well.

In order to understand the immunomodulatory potential of the compound of Formula E mRNA expression profiling of IFNγ, IL4, IL17A genes against the HGPRT endogenous control in the splenocytes samples from different groups were carried out. Data indicates dramatic inhibition in IL4 across all the groups and inhibition of IL17A expression in δI/200 and δIII/p400 (FIG. 6B). Interestingly, δI/200 showed the highest induction of INFγ across all groups.

Histological Assessment of Lung Pathology Associated with SARS-CoV2 Infection in the Compound of Formula E Treated Hamsters.

To understand the effect of administration of the compound of Formula E in terms of lung pathology upon SARS-CoV2 infection; detailed histological analysis of the lung samples isolated at 4 days post infection was carried out.

Histological assessment of the lung pathology was carried out by H&E staining in the compound of Formula E administered hamsters and compared with the control groups. FIG. 7 shows the histological images of H&E stained lungs at 40× magnification showing regions of pneumonitis (blue arrow), inflammation (black arrow), lung injury (red arrow), alveolar epithelial cells (green arrow) and their respectively pathological score and overall disease score for lung samples.

The H&E stained lung samples of all drug treated group showed good reduction in pneumonitis, alveolar epithelial injury and lung inflammation score. δIII/p400 group gave the best protection for all the histological parameters studied and the overall disease score. Interestingly, there was little, or no protection observed with δIV/400 drug group as compared to the infection control.

### Conclusion

SARS-CoV-2 challenge study in golden Syrian hamster indicated that out of the 4 dosing regimen tested pre-treatment with 400 mpk of the compound of Formula E showed best overall protective efficacy against SARS-CoV-2 infection giving decreased lung viral load and pneumonitis compared to Remdesivir. It has also exhibited decreased lung pathology and suppression of pathogenic IL4 and IL17A immune response.

The above tests indicate the use of the compound of Formula I was effective in reducing the V-ATPase in the cells and thereby helped in treating viral infection by inhibition of SARS CoV-2 virus. The compounds of Formula I and particularly compound of Formula E was highly effective as an anti-viral agent in treating viral infections without causing toxicity.

The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the disclosure.

1. A compound of Formula I for inhibiting V-ATPase activity in a cell, 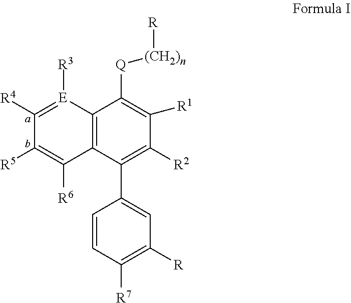
wherein,
E is selected from C, N;
Q is O, —NH;
n is 0-6;
R¹ and R² is selected from —H, —C(O)O-alkyl such as —C(O)OC2H5, or R¹ and R² together form a substituted or unsubstituted 5- or 6- membered ring such as lactone;
R³, R⁴, R⁵and R⁶ each independently is selected from —H, —OH, alkoxy;
R⁷ and R⁸ is selected from —H, —OH, alkoxy, —X where X can be F, Cl, Br or R⁷ and R⁸ together form a 5 membered ring containing one or more heteroatoms such as O;
a and b denote the presence or absence of double bond at the respective positions;
R is selected from substituted or unsubstituted phenyl ring, 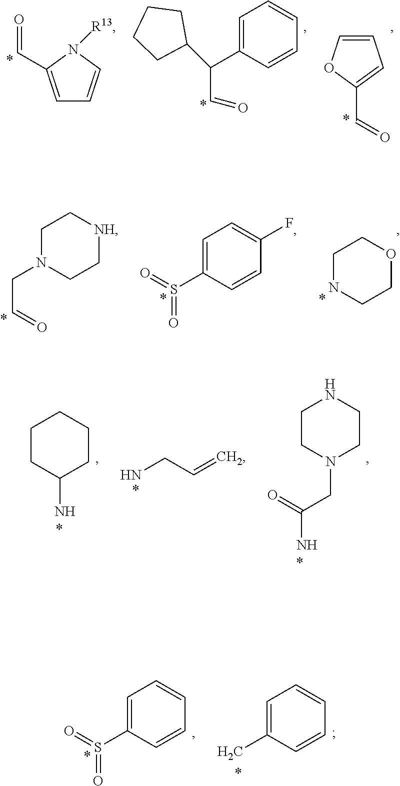
Wherein R⁹, R¹⁰, R¹¹ and R¹² each independently is selected from —H, —OH, alkoxy or R¹¹ and R¹² can be substituted or unsubstituted 5- or 6- membered ring such as lactone, —C(O)O-alkyl;
wherein R¹³ is H, alkyl; and
* represents linkage with Q or —CH2 group.

2. The use of the compound as claimed in claim 1, wherein the compound is of Formula II, 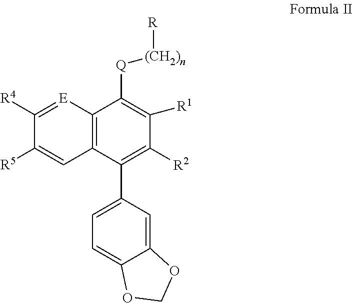
wherein,
E is selected from C, N;
Q is O, —NH;
n is 0-6;
R¹ and R² is selected from —H, —C(O)O-alkyl, or R¹ and R² together form a substituted or unsubstituted 5- or 6- membered ring such as lactone;
R⁴ and R⁵ each independently is selected from —H, —OH, alkoxy;
R is selected from substituted or unsubstituted phenyl ring, 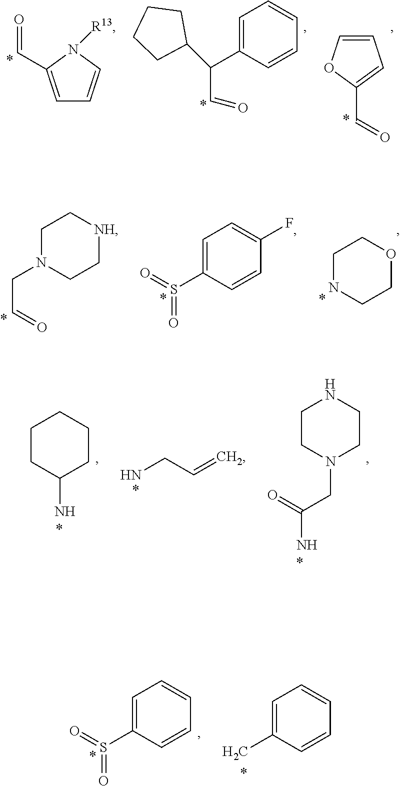
wherein R⁹, R¹⁰, R¹¹ and R¹² each independently is selected from —H, —OH, alkoxy;
wherein R¹³ is H, alkyl; and
* represents linkage with Q or —CH2 group.

3. The compound as claimed in claim 1, wherein the compound is of Formula III, 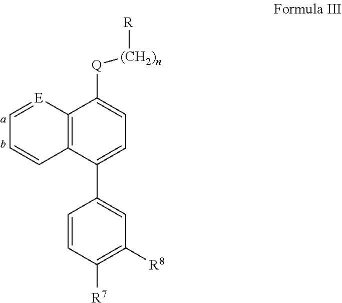
wherein,
E is selected from C, N;
Q is O, —NH;
n is 0-6;
R⁷ and R⁸ is selected from —H, —OH, alkoxy, —X where X can be F, Cl, Br or R⁷and R⁸ together form a 5 membered ring containing one or more heteroatoms such as O;
a and b denote the presence or absence of double bond at the respective positions;
R is selected from substituted or unsubstituted phenyl ring, 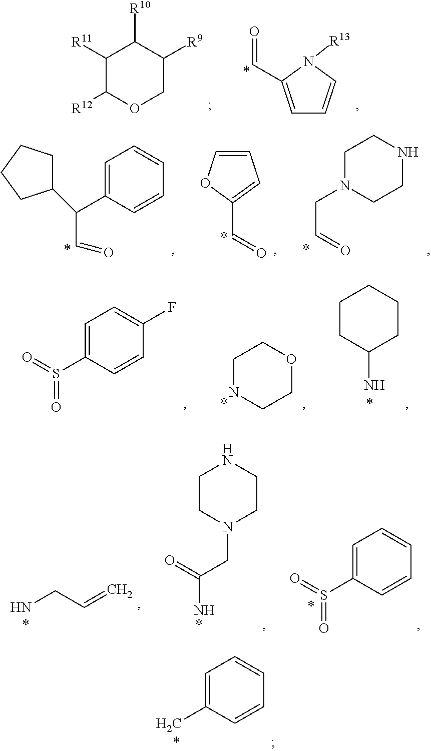
wherein R⁹, R¹⁰, R¹¹ and R¹² each independently is selected from —H, —OH, alkoxy;
wherein R¹³ is H, alkyl; and
* represents linkage with Q or —CH2 group.

4. The compound as claimed in claim 1, wherein the compound is

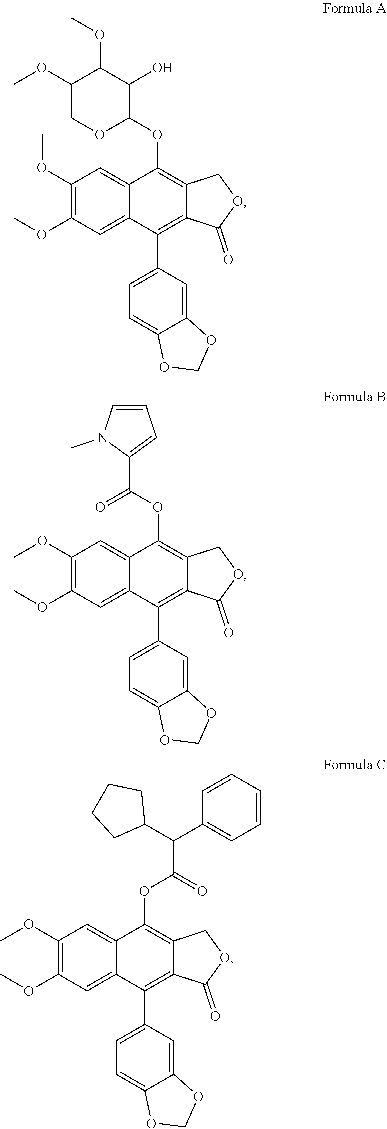

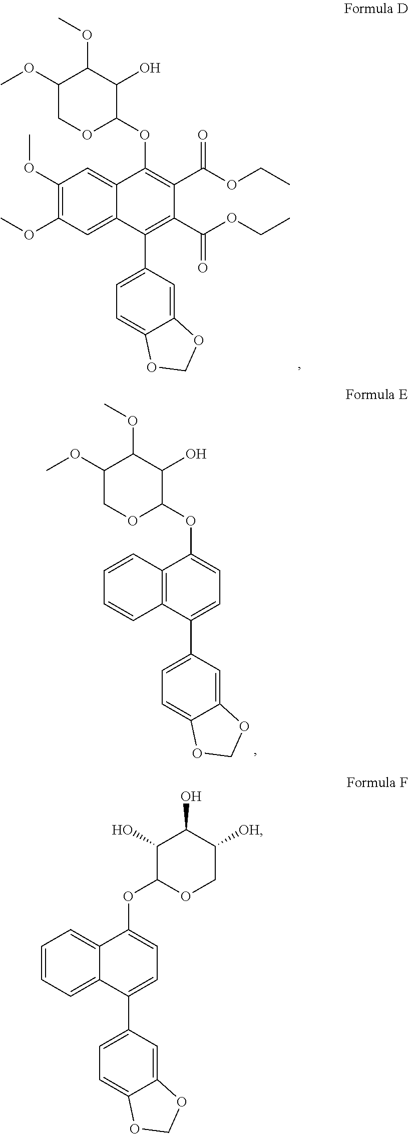

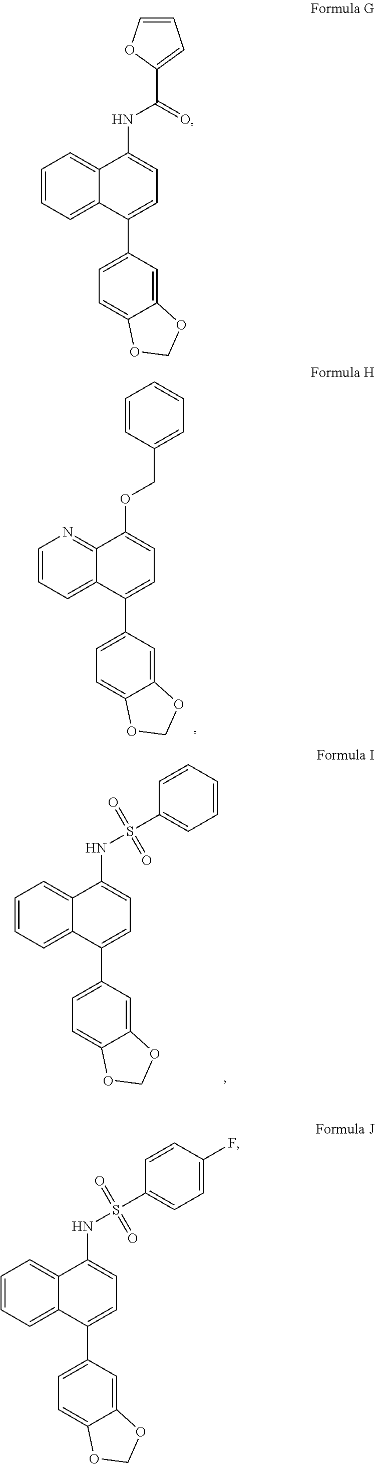

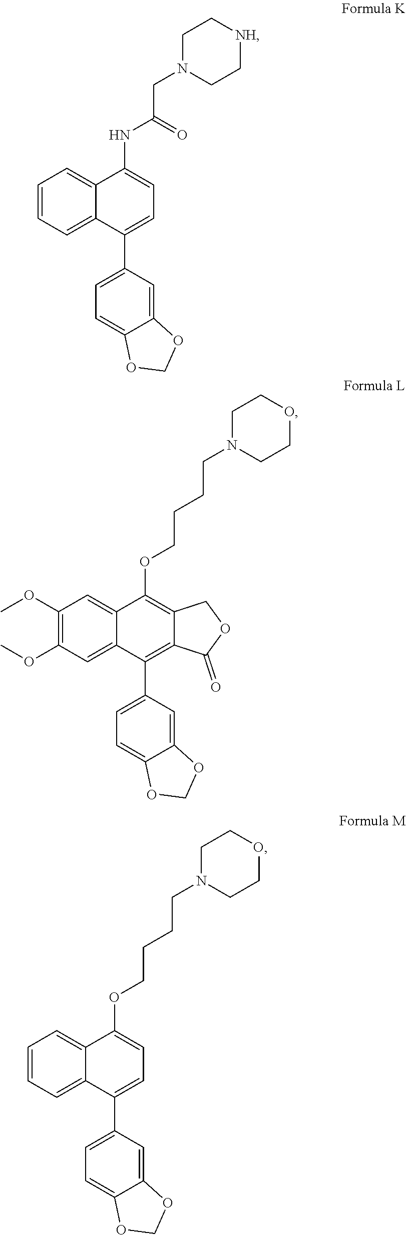 [SMILES: O=C(CN1CCNCC1)Nc1ccc(-c2ccc3c(c2)OCO3)c2ccccc12 ; COc1cc2c(OCCCCN3CCOCC3)c3c(c(-c4ccc5c(c4)OCO5)c2cc1OC)C(=O)OC3 ; c1ccc2c(-c3ccc4c(c3)OCO4)ccc(OCCCCN3CCOCC3)c2c1]

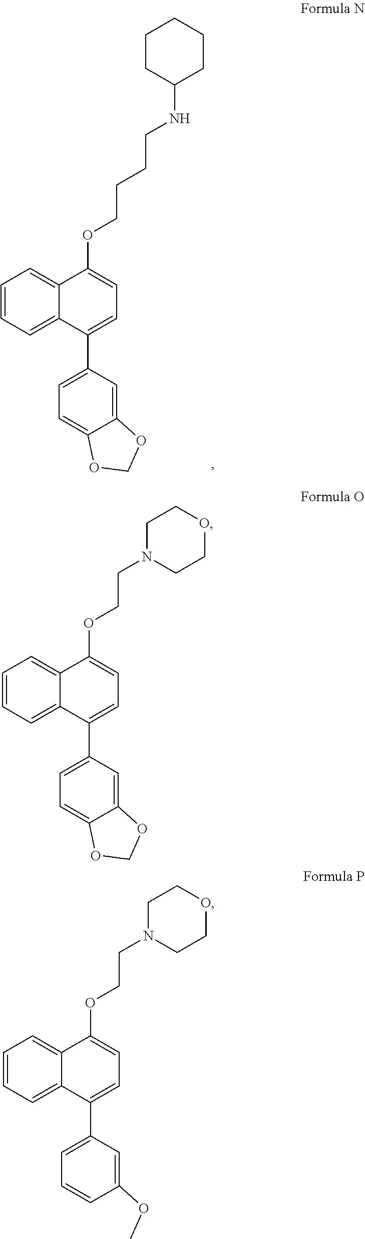 [SMILES: c1ccc2c(-c3ccc4c(c3)OCO4)ccc(OCCCCNC3CCCCC3)c2c1 ; c1ccc2c(-c3ccc4c(c3)OCO4)ccc(OCCN3CCOCC3)c2c1 ; COc1cccc(-c2ccc(OCCN3CCOCC3)c3ccccc23)c1]

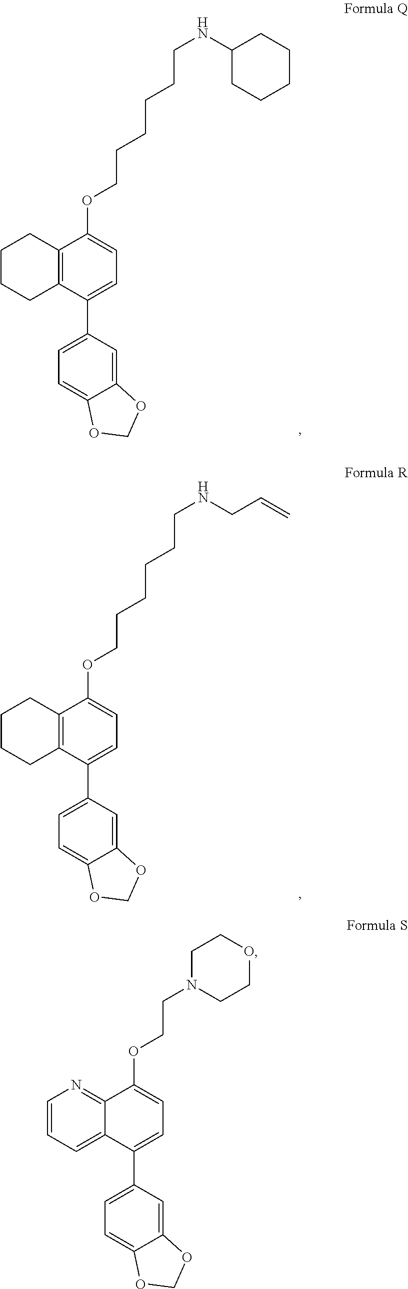 [SMILES: c1cc2c(cc1-c1ccc(OCCCCCCNC3CCCCC3)c3c1CCCC3)OCO2 ; C=CCNCCCCCCOc1ccc(-c2ccc3c(c2)OCO3)c2c1CCCC2 ; c1cnc2c(OCCN3CCOCC3)ccc(-c3ccc4c(c3)OCO4)c2c1]

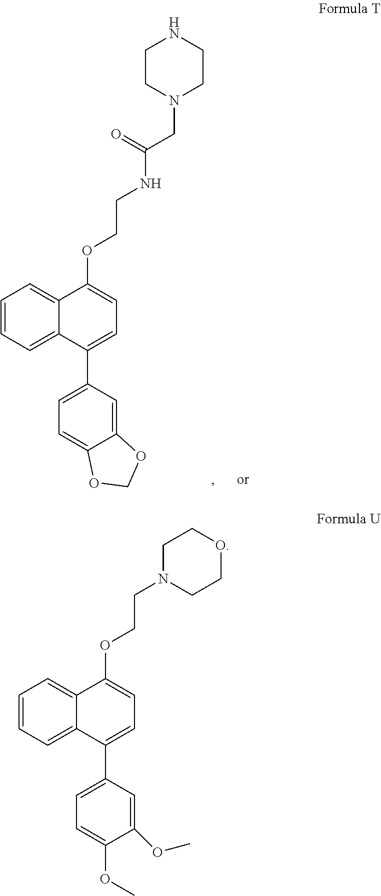 [SMILES: O=C(CN1CCNCC1)NCCOc1ccc(-c2ccc3c(c2)OCO3)c2ccccc12 ; COc1ccc(-c2ccc(OCCN3CCOCC3)c3ccccc23)cc1OC]

5. A compound of Formula E for inhibition of V-ATPase activity in a cell

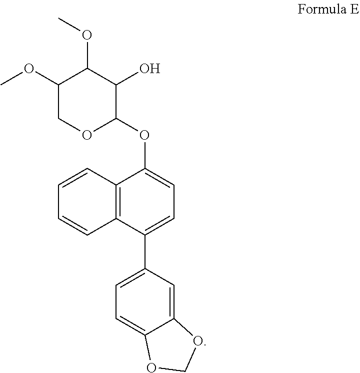 [SMILES: COC1COC(Oc2ccc(-c3ccc4c(c3)OCO4)c3ccccc23)C(O)C1OC]

6. The compound as claimed in claim 1, wherein, the compound is a prodrug, metabolite, pharmaceutically acceptable salt, solvate or polymorph thereof.

7. The compound as claimed in claim 1, for inhibition of a virus by inhibiting V-ATPase activity in a cell.

8. The compound as claimed in claim 7 for inhibition of SARS-CoV-2 virus or influenza virus.

9. A compound of Formula I for inhibition of SARS-CoV-2 virus or influenza virus.

10. U A compound of Formula E for inhibition of SARS-CoV-2 virus or influenza virus.

11. A method of inhibition of V-ATPase activity in a cell comprising contacting the cell with at least one or more compounds as claimed in claim 1.

12. A method of inhibition of a virus comprising contacting the virus infected cell with at least one or more compounds as claimed in claim 1.

13. The method of inhibition as claimed in claim 12 wherein, the virus is SARS-CoV-2 virus or influenza virus.

14. The method of inhibition as claimed in claim 1 wherein, the compound is of Formula E.

15. A method of treatment of a viral infection comprising administering a therapeutically effective amount of one or more of the compounds as claimed in claim 1 to inhibit SARS-CoV-2 virus or influenza virus.

16. The method of treatment as claimed in claim 15 wherein, the compound as claimed in claim 1 is combined with at least one additional compound having V-ATPase inhibitory activity.

17. The method of treatment as claimed in claim 15 wherein, the compound is of Formula E.

18. A compound of Formula I for preparing a medicament to inhibit V-ATPase activity in a cell for treatment of viral infections.

19. The method of inhibition as claimed in claim 12 wherein, the compound is of Formula E.

20. The method of treatment as claimed in claim 16 wherein, the compound is of Formula E.
